(19) United States
(12) Patent Application Publication
(10) Pub. No.: US 2005/0100967 A1
(43) Pub. Date: May 12, 2005

(54) Detection of endometrial pathology

(75) Inventors: Kimberly K. Leslie, Albuquerque, NM (US); Charlotte D. Mobarak, Albuquerque, NM (US); Harriet O. Smith, Albuquerque, NM (US)

Correspondence Address: MUETING, RAASCH & GEBHARDT, P.A., P.O. BOX 581415, MINNEAPOLIS, MN, 55458, US

(73) Assignee: SCIENCE & TECHNOLOGY CORPORATION @ UNM, Albuquerque, NM (US)

(21) Appl. No.: 10/888,180

(22) Filed: Jul. 9, 2004

Related U.S. Application Data
(60) Provisional application No. 60/559,932, filed on Apr. 6, 2004.
(60) Provisional application No. 60/486,528, filed on Jul. 11, 2003.

(51) Int. Cl. G01N 33/53; G06F 19/00; G01N 33/48; G01N 33/50

(52) U.S. Cl. 435/7.1; 436/86; 702/19

(57) ABSTRACT

Biomarkers and polypeptide profiles for screening, detection, and monitoring of endometrial pathology, including endometrial cancer.

This application claims the benefit of U.S. Provisional Applications Ser. Nos. 60/486,528, filed Jul. 11, 2003, and 60/559,932, filed Apr. 6, 2004, each of which is incorporated herein by reference in its entirety.

### STATEMENT OF GOVERNMENT RIGHTS

This invention was made with government support under grants from the National Institutes of Health, Grant Nos. 1-R24CA883399 and 1-R01CA99908-1. The U.S. government has certain rights in this invention.

### BACKGROUND OF THE INVENTION

Endometrial cancer is the most frequent invasive gynecologic malignancy and the fourth leading cause of cancer in women. When detected early, outcomes are favorable; nevertheless of the approximately 39,000 new cases of endometrial cancer reported annually in the United States, nearly 7,000 women die of advanced disease. Lifetime endometrial cancer risk in the US is 2.4%. Endometrial cancer has been identified by the National Cancer Institute as an under-studied disease by the recent Progress Review Group for Gynecologic Cancers. Early diagnosis leading to surgical cure by hysterectomy is the mainstay of current therapy.

Endometrial cancer is primarily a sporadic disease driven by complex interactions between somatically acquired genetic lesions (P53, PTEN, KRAS, microsatellite instability) and ambient hormonal selection factors. A very small fraction, less than 5% of endometrial cancers occurring in young women, present as a manifestation of multi-cancer heritable syndromes such as hereditary nonpolyposis colon cancer (HNPCC). The majority of endometrial cancers are discovered when the patient develops symptomatic bleeding, followed by a diagnostic endometrial biopsy. Under these circumstances, 21% of endometrial adenocarcinomas at the time of initial diagnosis have already extended beyond the subjacent myometrium, having extended to the cervix (Stage 2, 5.8%), regional nodes or extrauterine tissues (Stage 3, 7.7%), or distant sites (Stage 4, 8.3%). If detected earlier, many of these patients could achieve surgical cure by hysterectomy alone.

There are currently no routine screening tests of practical utility in detection of endometrial cancer. Endometrial biopsies and curettings cannot be considered a primary screening tool because they are invasive, can cause cramping and bleeding, and carry risks of uterine perforation or contamination of the cavity by pathogens. Biopsies are thus reserved for symptomatic or very high risk (such as those women with HNPCC) patients. Less intrusive are routine PAP smears, which do not transgress the uterine cavity directly. Rarely, a cytopathologist examining PAP smears intended to detect cervical disease will incidentally recognize malignant endometrial cells in the specimen. Cytologic evaluation of PAP smears is, however, an insensitive means of endometrial cancer detection and for this reason is not recommended. Transvaginal ultrasound has also been evaluated as a possible screening tool for endometrial carcinoma. Cancer detection sensitivity for transvaginal ultrasound with a threshold endometrial thickness of 6 mm is only 17%; and 33% for a threshold value of 5 mm. Specificity is very low, making this an expensive (during follow up of numerous false positives) as well as insensitive test.

Pre-cancerous and other benign endometrial lesions, such hyperplasia and endometriosis, also pose a significant health risk for women, and convenient screening tests are not available to diagnose these conditions.

Proteomics represents the effort to establish the identities, quantities, structures, and biochemical and cellular functions of all proteins in an organism, organ, or organelle, and how these properties vary in space, time, or physiological state. Proteins serve to relay the physiological status of a cell during various phases of a disease. Although this topic has been studied for many decades, in the past this has been done mostly on a one-protein-at-a-time basis. The human proteome contains potentially thousands of intact and cleaved proteins. Using proteomic techniques, changes in proteins that are overexpressed and shed into body fluids can be examined as unique patterns. These patterns can be reflective and diagnostic of a given disease state.

At the emerging interface between clinical medicine and proteomics, methodologies have been developed to identify patterns of biomarkers having clinical relevance. It is now being recognized that the diagnostic endpoint for disease detection may not be a single analyte, but a proteomic pattern that is composed of many individual proteins, each of which individually cannot differentiate diseased from healthy individuals.

High throughput proteome-wide technologies such as surface enhanced laser desorption and ionization with time of flight detection (SELDI-TOF), or liquid-chromatography-tandem mass spectroscopy (LC-MS/MS) can be used to generate proteomic fingerprints from serum and tissue samples which are specific for disease. In SELDI, analytes are captured onto a substrate surface, which typically takes the form of a microchip array. Von Eggeling, et al. reported the utilization of ProteinChip (Ciphergen Biosystems, Inc., Fremont, Calif.) microarray technology as a platform for SELDI mass spectrometric for the analysis of cancerous tissue protein profiles (2000, BioTechniques 29: 1066-1070). That study described the use of protein microarray analysis for distinguishing between cancerous and normal tissue. There are numerous other reports on the utilization of protein microarray technology for the identification of candidate genes involved in tissue repair/regeneration, disease diagnosis, as well as cancer biomarker identification, further supporting the role of high-through put protein analysis in research and clinical settings. Recently, serum-based proteomic pattern analysis has been used to diagnosis ovarian cancer (Vlahou et al., 2003, J Biomed Biotechnol 2003, 308-314; Petricoin et al., 2002, Lancet 359, 572-577). Other examples of the use of SELDI-TOF to perform proteomic analysis on tissue or serum samples include Li et al. (2000, Biochim. Biophys. Acta 1524: 102-109); Tonge et al. (2001, Proteomics 1: 377-396); Vlahou et al. (2001, Am. J. Pathol. 158: 1491-1502); Reddy et al. (J. Biomed. Biotechnol. 2003, 2003(4):237-241); Wright, Jr. (Expert Rev. Mol. Diagn. 2002, 2(6):549-563); Wright, Jr. et al. (Prostate Cancer Prostatic Dis. 1999, 2(5-6):264-276); Paweletz et al. (Drug Dev. Res. 2000 49:34-42); Cazares et al. (prostate cancer, Clin. Cancer Res. 2002, 8(8):2541-2552); and Paweletz et al. (breast cancer, Dis. Markers 2001, 17(4):301-307).

Advances in artificial intelligence have yielded bioinformatics programs that can apply pattern recognition systems with iterative clustering and survival of the fittest analysis to yield highly discriminative diagnostic algorithms. For example, Petricoin and colleagues (2002, Lancet 359, 572-577) used mass spectroscopy to generate proteomic spectra from patients with and without ovarian cancer. Using a genetic algorithm, they found a cluster pattern, which could segregate cancer cases from non-malignant ones with a sensitivity of 100% and a specificity of 95%.

Without convenient and easily accessible screening tests for cancer, diagnostic delays will continue to plague the health care system and thwart efforts to detect and treat malignancies in their earliest stages. Endometrial cancer markers that are shed locally (into the uterine lumen, and ultimately through the cervix into the vagina) or systemically (into blood) would present a readily accessible fluid format for proteomics-based early detection. The development of a non-invasive, proteomics-based screening test for endometrial pathology would represent a significant medical advance.

### SUMMARY OF THE INVENTION

The present invention makes possible the rapid and noninvasive evaluation of endometrial pathology in a subject. The method is useful for evaluating the presence, absence, nature and/or extent of an endometrial pathology. Endometrial pathology can include, without limitation, endometrial cancer, hyperplasia or endometriosis. A body fluid or tissue, such as blood, serum, plasma, or vaginal secretions, is examined to evaluate protein expression. A plurality of polypeptides is detected in the biological sample (test sample) obtained form the patient to yield a protein profile for the test sample. The test protein profile is compared to a reference protein profile, and an observed difference is indicative of the presence, absence, nature or extent of the endometrial pathology in the patient. The reference protein profile reflects a known disease state (e.g., endometrial cancer, endometriosis, or a normal control) and preferably includes one or more biomarker polypeptides associated with endometrial pathology. In a preferred embodiment, the difference between the test protein profile and the reference protein profile comprises a difference in the amount of at least one biomarker polypeptide represented by a M/Z peak value in Tables 3, 4, 5, 6 or 7.

The method for evaluating endometrial pathology in a subject can include discriminating between different disease states or between a disease state and normal state. It can also be used to monitor the extent of the progression or regression of endometrial disease, such as cancer, in a given patient. To this end, the reference protein profile can be derived from a sample previously obtained from the patient, for example a sample obtained prior to treatment or as part of a general health screening. The method is thus well-suited to evaluate the efficacy of treatment decisions, such as drugs or surgeries.

Optionally, the method further comprises designing a classification model or algorithm, or enhancing or refining an existing classification model or algorithm, based on at least one difference between the test protein profile and the reference protein profile.

The method for evaluating the presence, absence, nature or extent of an endometrial pathology in a patient can, alternatively or additionally, involve a comparison of the patient's test protein profile (or various components thereof) with predetermined reference values for one of more biomarker for endometrial pathology. In this embodiment, the method includes providing a biological test sample obtained from the patient; detecting a plurality of polypeptides in the test sample to yield a test protein profile showing the amount of at least one biomarker polypeptide in the sample; and comparing the amount of the biomarker polypeptide in the sample with at least one predetermined reference value. The difference between the amount of the biomarker polypeptide in the sample and the predetermined reference value is indicative of the presence, absence, nature or extent of the endometrial pathology in the patient.

Advantageously, the test protein profile can be generated using mass spectrometry. The polypeptides are preferably detected using surface-enhanced laser desorption/ionization time of flight (SELDI-TOF) mass spectrometry, and the amount of the biomarker polypeptide is indicated as a spectral peak intensity. The method optionally includes immobilizing the plurality of polypeptides on a microarray prior to detecting the polypeptides.

In another embodiment, the method for evaluating the presence, absence, nature or extent of an endometrial pathology in a patient involves the evaluation of a test protein profile of the patient without the use of a reference protein profile, using instead an internal standard. This embodiment of the invention involves detecting at least one biomarker polypeptide in the patient's test sample; detecting at least one reference polypeptide in the test sample as well; comparing the amount of the biomarker polypeptide to the amount of the reference polypeptide in the test sample to yield a test value; and comparing the test value to a predetermined reference value. The difference between test value and the predetermined reference value is indicative of the presence, absence, nature or extent of the endometrial pathology in the patient.

In preferred embodiments of the invention, the biomarker polypeptide is represented by a M/Z peak value in Tables 3, 4, 5, 6 or 7 (Example I).

In another embodiment of the method for evaluating the presence, absence, nature or extent of an endometrial pathology in a patient, the patient's test protein profile is analyzed using a classification model or algorithm to discriminate the presence, absence, nature or extent of the endometrial pathology in the patient. This analysis is preferably performed with the assistance of a computer. The model or algorithm is derived from analysis of a plurality of protein profiles known to be associated with the presence, absence, nature or extent of the endometrial pathology. The analysis can be made using supervised or unsupervised learning methods. Preferably, the analysis is made using a recursive partitioning process, such as a decision tree classification model. In a preferred method, the model or algorithm discriminates on the basis of the presence, absence or amount of at least one biomarker polypeptide having m/z listed in Tables 3, 4, 5, 6 and 7 (Example I).

In another aspect, the invention provides a computer-assisted method for evaluating the presence, absence, nature or extent of an endometrial pathology in a patient. The method includes providing a computer comprising model or algorithm for classifying data from a biological sample obtained from a subject, wherein the classification includes analyzing the data for the presence, absence or amount of at least one biomarker polypeptide; inputting data from a biological sample obtained from a subject; and classifying the biological sample to indicate the presence, absence, nature or extent of an endometrial pathology. Preferably, the method of claim 24 wherein the biomarker polypeptide is represented by a M/Z peak value in Tables 3, 4, 5, 6 or 7 (Example I).

In another aspect, the invention provides a method for identifying a polypeptide biomarker associated with the presence, absence, nature or extent of an endometrial pathology, as well as biomarkers thus identified and described herein (see Tables 3, 4, 5, 6 or 7 in Example I, and FIG. 2). In one embodiment, comparison of a test protein profile with a reference protein profile permits identification of a biomarker polypeptide associated with the presence, absence, nature or extent of endometrial pathology in the patient. In another embodiment, the method includes (a) providing a first plurality of biological samples obtained from test patients known to be afflicted from an endometrial pathology; (b) providing a second plurality of biological samples obtained from control patients known to be free of the endometrial pathology; (c) detecting a plurality of polypeptides in the first and second plurality of samples to yield test and control protein profiles; and (d) comparing the test and control protein profiles to identify a polypeptide biomarker associated with the presence, absence, nature or extent of the endometrial pathology.

Optionally, the polypeptide biomarker thus identified is isolated and characterized. The amino acid sequence can be determined. The polypeptide biomarker can be evaluated for its suitability as a therapeutic target. If the polypeptide biomarker is determined to be a potential therapeutic target, the method optionally further includes screening candidates compounds for efficacy in altering the bioactivity of the biomarker polypeptide.

The present invention thus provides a useful method for detecting biomarker polypeptides associated with endometrial pathology. A protein profile obtained from a biological sample of a subject suspected of having an endometrial pathology is compared to a reference protein profile (or a reference value for one or more biomarker components of the reference protein profile), and polypeptides that are differentially expressed polypeptides between the first and second profiles are detected. The presence, absence, nature or extent of an endometrial pathology in a patient can be evaluated in view of the expression of at least one differentially expressed biomarker polypeptide, and/or a biomarker polypeptide can be isolated and identified.

In yet another aspect, the invention provides a method for screening a patient or population of patients for endometrial pathology by assaying for the presence of at least one biomarker polypeptide associated with endometrial pathology in a sample obtained from a patient. The biomarker polypeptide is preferably one that is represented by a M/Z peak value in Tables 3, 4, 5, 6 or 7 (Example I). The assay can be a mass spectrometric assay but advantageously can also be an immunoassay, such as a Western blot or an enzyme linked immunosorbent assay (ELISA). A plurality of biomarker polypeptides can be analyzed, thereby increasing the predictive power of the screening assay.

### BRIEF DESCRIPTION OF THE DRAWINGS

FIG. 1 shows representative spectra obtained by surface enhanced laser desorption ionization time of flight (SELDI). Panels A and B each show a representative serum spectrum from an endometrial cancer patient and a normal control. Spectrum view and pseudo gel view are both shown. Panel A shows an example of peaks that have lower expression levels on average from patients with cancer compared with the serum from controls. Panel B shows an example of peaks that have higher expression levels on average from patients with cancer compared with the serum controls.

FIG. 2 shows decision tree classification models generated using an H50 microarray (Ciphergen, Fremont, Calif.) on non-fractionated serum (A); and an IMAC30 microarray (Ciphergen, Fremont, Calif.) for various serum fractions (B) pH<5; (C) pH 5-7; (D) pH>7; and (E) non-fractionated.

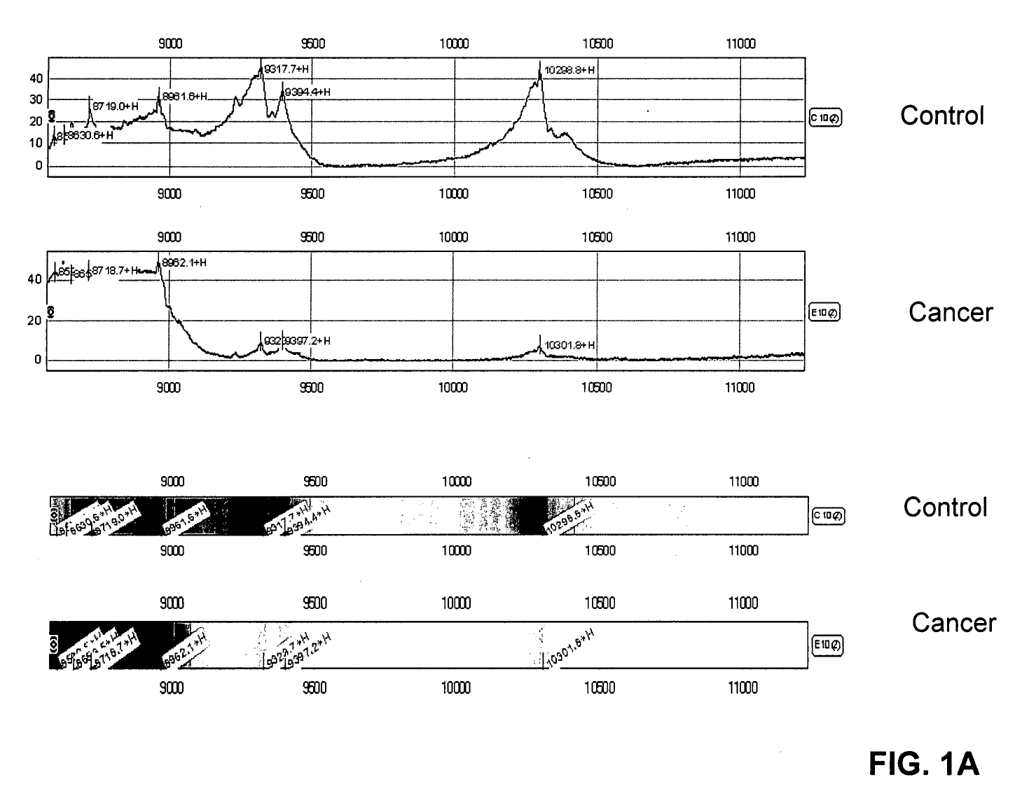

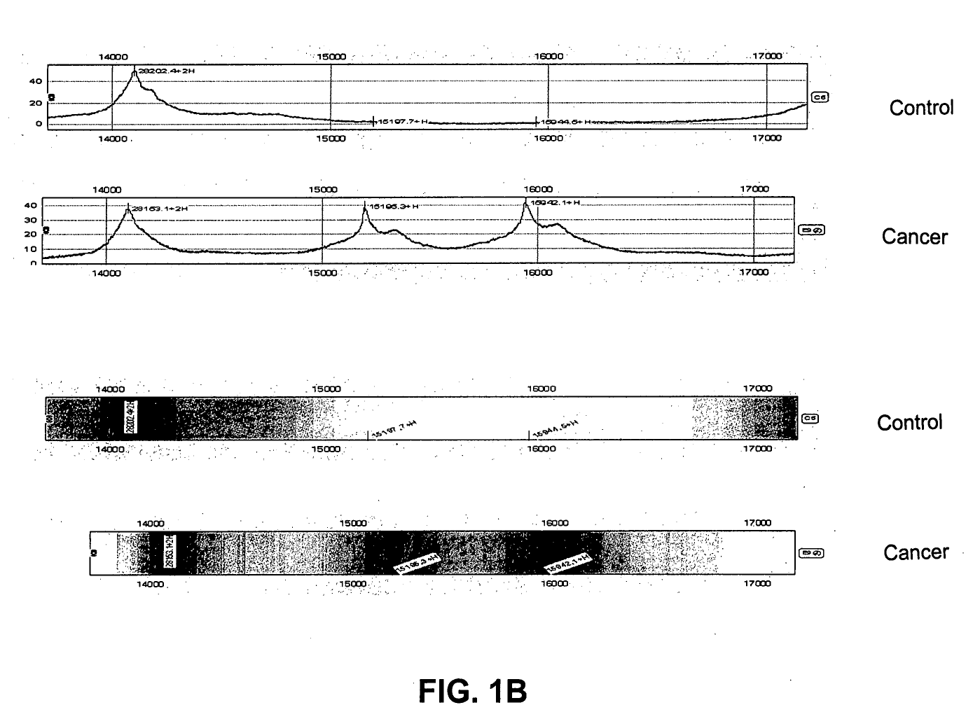

### DETAILED DESCRIPTION OF ILLUSTRATIVE EMBODIMENTS

The invention provides a non-invasive screening test for endometrial pathology, including endometrial cancer. It also significantly enhances the detection of asymptomatic disease. The method of the invention is useful to detect and monitor endometrial cancer, pre-cancer (such as endometrial hyperplasia or any type), endometriosis, or other diseases of the endometrium. The method also facilitates identification of those women at risk for cancer, those with existing, undetected cancer, those with cancer who will suffer a recurrence, and those with other diseases of the endometrium including hyperplasia and endometriosis. When used to detect endometrial cancer, the invention is well-suited not only to diagnosis, but also to predict extrauterine spread and response to therapy.

The invention addresses a major impediment to the management of endometrial carcinoma that continues to result in treatment failures: the diagnosis may be made late in the process of carcinogenesis. It provides a sensitive and specific non-invasive screening test for endometrial cancer and other endometrial conditions. Additionally, when used as an at-large screening tool, the method of the invention can reduce the need for painful and expensive endometrial biopsy.

The invention provides biomarker patterns, preferably serum biomarker patterns, that are indicative of endometrial pathology, particularly cancer. These patterns not only facilitate detection endometrial pathology, such as cancer, in its early stage, but also facilitate design of therapeutic targets and patient-tailored therapy.

Analysis of a patient's protein profile can be used for diagnostic or prognostic purposes. Biomarker analysis can be used to design a therapeutic plan for a patient, and to provide a measure of the success of the plan over time. Polypeptide biomarkers represent a convenient method for evaluating clinical trials, and may provide a basis for drug development as particular biomarkers are identified and characterized.

It is noted here that as used in this specification and the appended claims, the singular forms “a,” “an,” and “the” include plural reference unless the context clearly dictates otherwise.

Polypeptide Biomarker

The present invention involves the identification and use of polypeptide biomarkers that are indicative of endometrial pathology. A polypeptide biomarker can include a peptide, polypeptide, protein, glycoprotein, phosphoprotein, lipoprotein and the like. The polypeptide biomarker can represent a known polypeptide or an unknown polypeptide. The method of the invention can be used to detect an intact polypeptide biomarker or a component thereof, such as a peptide component, of the constituent components of the polypeptide produced by proteolysis, glycolysis, lipidolysis and the like. When mass spectrometry is used to analyze a sample, the presence of a polypeptide associated with endometrial pathology is evidenced by one or more peaks in a spectrum, each peak characterized by a particular mass to charge (m/z) ratio.

If the polypeptide biomarker corresponds to a polypeptide that is already known or subsequently identified, it can serve as a therapeutic target as described in more detail below. If it represents an unknown polypeptide, it is still useful for indicating the presence or absence of disease. Multiple biomarker polypeptides are shown herein to be associated with endometrial pathology, and the invention includes analyzing any subset of these proteins to assess endometrial pathology.

It should be understood that the terms “biomarker”, “polypeptide biomarker” and “biomarker polypeptide” can, depending on the context, refer to the physical polypeptide itself or to a graphical or numerical representation of the polypeptide such as a peak in a mass spectrum trace, a band on a gel image, a numerical value, and the like. For example, a particular M/Z value or peak in a SELDI-TOF spectrum may be referred to as “biomarker” for endometrial pathology. This graphical or numerical “biomarker” reflects the existence of the underlying expressed polypeptide biomarker in the test sample which gave rise to the protein profile. The underlying expressed polypeptide biomarker can be detected in any convenient way.

Biological Sample

The biological sample can include any body fluid or tissue. Preferred body fluids include blood, plasma, serum, urine, saliva, sputum, cerebrospinal fluid, mucus, and vaginal and rectal secretions; preferably the biological sample includes blood or blood products such as plasma and serum. As the invention is directed toward the analysis of endometrial pathology, endometrial tissue is a preferred tissue sample however the method can be used to analyze other female reproductive tissue as well including tissue from the uterus, cervix, vagina and the like. When tissue samples are used, such as biopsies, they can be homogenized, for example in phosphate buffered saline or, alternatively, in a detergent-containing buffer to solubilize the polypeptides to be detected.

It should be noted that although the invention is described primarily with respect to endometrial pathology in humans, it is equally application to all mammalian subjects and, in that regard, has application in veterinary as well as human medical contexts.

Sample Processing

Optionally, the test sample can be preprocessed prior to analysis of its protein content, for example to remove nonproteinaceous sample components. Methods for preprocessing include, without limitation, various forms of chromatography (size exclusion, hydrophobic, ion exchange, affinity and the like), microfiltration, centrifugation and dialysis. Preprocessing also can include subjecting the sample to chemical or enzymatic protein cleavage agents in order to break down the proteins into smaller components. Additionally or alternatively, the test sample is optionally fractionated into subsamples, each containing a subset of sample proteins, prior to analyzing the sample for polypeptide biomarkers.

The amount a biomarker polypeptide in the test sample or a control sample can be zero, in which case “amount” refers to the presence or absence of the protein, which presence or absence is indicative of endometrial pathology. Alternatively, the biomarker polypeptide can be present in both samples, but at a higher (upregulated) or lower (downregulated) level in the test sample which is indicative of endometrial pathology.

Amounts of biomarker polypeptides can be determined in absolute or relative terms. If expressed in relative terms, amounts can be expressed as normalized amounts with reference to a selected protein present in the sample.

In some embodiments of the invention, after optional preprocessing and/or fractionation, proteins are physically separated prior to determining the amounts of each protein. Physical separation can be achieved, for example, using single or multidimensional chromatography, electrochromatography or electrophoresis, such as 2D electrophoresis. The amount of the separated proteins can be determined using any convenient method such as spectroscopic (e.g., UV detection) or colorimetric (e.g., staining) methods. Optionally, the identity of separated proteins of interest can be determined using standard techniques such as protein sequencing and tandem mass spectrometry.

In other embodiments of the invention, after optional preprocessing and/or fractionation, sample components are not further separated but instead the sample is subjected to mass analysis, for example using peptide-mass fingerprinting or mass spectrometry.

Polypeptide Immobilization

In a preferred embodiment, a protein profile for the test sample is obtained using mass spectrometric analysis. Protein microarray technology is particularly well-suited for use in this embodiment of the invention. Microarrays of capture agents bind to proteins in the sample, facilitating analysis of the amount of the bound proteins, particular in mass spectrometry applications. Materials suitable for use as microarray surfaces include polymeric materials and plastics, particularly organic polymers; silica-based substrates such as glass, quartz, silicon and polysilicon including silicon wafer; ceramic; metals; beads (porous or non-porous) of cross-linked polymers (e.g., dextran, agarose, etc.); composite materials; and the like. Optionally the microarray surface is coated with a material, for example, gold, titanium oxide, silicon oxide, etc. that allows derivatization of the surface. Suitable microarray surface chemistries, as well as other aspects of microarray capture and detection, are described in U.S. patent Pub. 20030232396, published Dec. 18, 2003 (Mathew et al.).

A microchip array may contain a chemically-treated surface, such as a cationic, anionic, hydrophobic or hydrophilic surface, or biochemically-treated surface, such as a surface comprising immobilized antibody, receptor, nucleic acids, etc., depending on the specific interaction desired to capture proteins of interest. In a preferred embodiment, proteins in a sample are bound to a chemically treated surface comprising, for example, an anion exchange agent, a metal affinity agent, or a hydrophobic (reverse phase) agent. Protein microchips produced by Ciphergen (Fremont, Calif.) contain surfaces having chromatographic or biospecific adsorbents attached thereto at addressable locations. A number of different surface chemistry capture agents are available in a microarray format on chips from Ciphergen. For example, carboxylate chemistry provides a negatively charged weak cation exchanger in the CM10 and WCX2 chips, and the SAX2 chip uses quaternary amine functionality for strong anion exchange. Ciphergen also sells chips with immobilized metal affinity capture agent (IMAC3 and IMAC30), an agent that mimics reversed-phase chromatography with C16 functionality (H4), and an agent that binds through reversed-phase or hydrophobic interactions (H50), among others. Each chemistry binds different proteins in a sample with differing degrees of selectivity. Unbound proteins are preferably removed by washing. The bound proteins can be referred to as the “retentate.”

Optionally, a single microarray chip can contain a plurality of spots with different capture agents. Alternatively or additionally, a sample can be analyzed using two or more microarrays with different chemistries, and the data combined to produce a classifier model as described more fully below.

In addition to Ciphergen (Fremont, Calif.), microarrays suitable for use in the invention are available from Packard BioScience Company (Meriden Conn.), Zyomyx (Hayward, Calif.) and Phylos (Lexington, Mass.).

Polypeptide Detection

In a preferred embodiment, immobilized polypeptides are detected using high throughput mass spectrometry, for example matrix-assisted laser desorption/ionization coupled with time-of-flight mass spectrometry (MALDI-TOF) or surface-enhanced laser desorption/ionization coupled with time-of-flight mass spectrometry (SELDI-TOF). See U.S. Pat. Nos. 5,719,060, 6,020,208, 6,027,942, 6,124,137, and 6,225,047 (all to Hutchens et al.). Mass spectrometry and associated methods for analysis of protein profiles (also known as “retentate maps”) are described in detail in U.S. patent Pub. 20040096820, published May 20, 2004, Rich et al.). The mass spectrometric matrix includes energy absorbing molecules that are capable of absorbing energy from a laser desorption/ionization source and thereafter contributing to desorption and ionization of analyte molecules in contact therewith. Example include cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others.

In matrix-assisted laser desorption/ionization (MALDI), the analyte is mixed with a solution containing a matrix, and a drop of the liquid is placed on the surface of a substrate. MALDI is a liquid phase method in which the matrix solution co-crystallizes with the analyte. The substrate is inserted into the mass spectrometer, and laser energy is directed to the substrate surface where it desorbs and ionizes the biological molecules without significantly fragmenting them. MALDI for large proteins is described in, e.g., U.S. Pat. No. 5,118,937 (Hillenkamp et al.) and U.S. Pat. No. 5,045,694 (Beavis et al.).

In surface-enhanced laser desorption/ionization (SELDI) the analyte is captured onto the substrate surface. In other words, the substrate surface is modified so that it is an active participant in the desorption process. SELDI is a solid phase method for desorption in which the analyte is presented on a surface that enhances analyte capture and/or desorption. The bound protein is bombarded with laser energy which induces its desorption from the surface and ionization.

In one version of SELDI, known as surfaced enhanced affinity capture (SEAC), the analyte is affinity-captured onto the substrate surface, and an energy absorbing matrix can be added to aid desorption. See, e.g., U.S. Pat. No. 5,719,060 (Hutchens et al.). In another version, known as surface enhanced neat desorption (SEND), a layer of energy absorbing molecules chemically bound to the substrate surface, and the sample is then applied to the surface. Like a matrix, the bound energy absorbing molecules assist in the desorption of the analyte. A version of SELDI that utilizes photolabile attachment molecules (surface-enhanced photolabile attachment and release, or SEPAR) can also be used. The photolabile attachment molecule is a divalent molecule having one site covalently bound to a solid phase and a second site that binds the affinity reagent or analyte.

A preferred SELDI system is the SELDI ProteinChip System available from Ciphergen Biosystems, Inc. (Fremont Calif.). Ciphergen's ProteinChip Arrays are analyzed in the ProteinChip Reader. The polypeptides are desorbed of the substrate surface, ionized, and detected using time-of-flight (TOF) mass spectrometry. Mass data is displayed as a spectrum trace that represents the proteins in the sample.

In both MALDI-TOF and SELDI-TOF, the time of flight of the ionized protein to a detector is recorded and converted to protein molecular weight (larger polypeptides generally have longer flight times). The amount and molecular weight of numerous proteins present in a sample can be detected simultaneously to generate a profile or spectrum of the proteins in the sample. With TOF-mass spectrometry, one can obtain information on hundreds or thousands of different proteins or peptides at a single site on an array. The method is capable of detecting nanomole to sub-femtomole quantities of protein on a spot, corresponding to millimolar to picomolar concentrations in a biological sample. Comparison of the profiles from different samples permits the identification of polypeptide differences between the samples, and the differences permit the assessment of the disease status in the test sample.

Alternatives to detection methods utilizing gas phase ion spectrometry (such as mass spectrometry) that can be used to produce a protein profile include optical detection methods such as fluorescence, phosphorescence, luminescence, chemiluminescence, absorbance, reflectance, transmittance, birefringence or refractive index. Optical methods further include without limitation surface plasmon resonance which detects binding events by using changes in the refractive index of a surface caused by increases in mass, resonance light scattering, ellipsometry, microscopy (both confocal and non-confocal), imaging methods and non-imaging methods. Optionally these methods are coupled with immunoassays, for example those that involve labeled secondary antibodies. Electrochemical methods (including voltametry and amperometry), radio frequency methods (including multipolar resonance spectroscopy), immunoassays (including ELISA), and atomic force microscopy are other examples of detection methods that can be used.

Polypeptide Analysis and Biomarker Identification

The series of peaks generated using mass spectrometry (or other polypeptide indicators generated using other detection mechanisms) constitutes a “protein profile” or “protein fingerprint” for that sample. The invention provides for the use of protein profiles or fingerprints, including individual biomarker constituents thereof, that have diagnostic or prognostic value for endometrial pathology, particularly endometrial cancer.

A “protein profile” is to be broadly understood to encompass one or more polypeptides in a sample. A protein profile can include one or more proteins, for example at least 10 proteins, at least 25 proteins, at least 100 proteins or at least 500 proteins. In some embodiments, the lower limit of the range of masses of the polypeptides profiled is at least 100 daltons, or 500 daltons, or 1,000 daltons. In some embodiments, the upper limit of the masses of the proteins profiled is at most 5,000 daltons, or 10,000 daltons, or 15,000 daltons, or 20,000 daltons, or 30,000 daltons, or 50,000 daltons. The protein profile can include the amount (including the presence or absence) of a single polypeptide biomarker, or the amounts (including the presence or absence) of two or more polypeptide biomarkers. The pattern of the presence and/or amount of polypeptides in a given sample, compared to a reference profile, can be used to generate a protein difference map. A protein difference map can be used to identify polypeptide markers that are up- or down-regulated (or present or absent) in the test sample. A protein difference map can also be used to identify trends in the amount of individual biomarkers, rather than absolute amounts of the biomarkers, that correlate with endometrial pathology.

Alternatively or additionally, ratios of the spectral intensities of various protein pairs can be analyzed instead of amounts or differences of the intensities. The use of ratios may yield a more sensitive measure of protein amounts or changes in amounts than protein difference maps.

In one embodiment, protein profiles are used to identify potential new biomarkers for endometrial pathology. The biomarker can be associated the presence, absence, nature or extent of an endometrial pathology. At least two populations of patients are identified: at least one test population characterized by a particular disease state, such as endometrial cancer or hyperplasia, and a second population which represents a control (disease-free) population. Protein profiles are obtained for members of both populations, for example from serum samples using SELDI-TOF. The test and control protein profiles reflect the presence and amounts of various protein components of the samples. Comparison of the protein profiles leads to the identification of a polypeptide biomarker associated with the presence, absence, nature or extent of the endometrial pathology. Optionally, composite, consensus or average profiles can be used in the comparison. As noted above, the observed polypeptide biomarkers may constitute one or more peptide components of a biomarker polypeptide if the sample is treated with a proteolytic agent prior to biomarker analysis.

In another embodiment, the presence, absence, or amount of one or more designated biomarkers in a protein profile is used to discriminate among different disease states, and/or to discriminate between disease and normal states. The protein profile for a test sample is compared to a reference protein profile. The reference profile includes polypeptide biomarkers for a control for which disease status is known. The control subject may be either free of disease, or afflicted with disease. The reference protein profile may represent a single subject or it may be an average, consensus or composite protein profile derived from samples from multiple subjects having the same disease state. Alternatively, predetermined numerical values, such as intensities, associated with one or more biomarkers in a test protein profile can be compared with reference values for the biomarkers, and deviations from the reference values may be indicative of disease state. Numerical values may represent raw, averaged or normalized values.

In another embodiment, the presence, absence or amount of one or more designated biomarkers in a particular profile is used to monitor the progression or regression of disease, for example in response to therapy. The protein profile of a test sample is compared to a reference protein profile. As described above, the reference protein profile may be a protein profile derived from a sample taken from subjects whose disease state is known. The subject may be either free of disease, or afflicted with disease. The reference protein profile may represent a single subject or it may be an average or composite protein profile derived from samples from multiple subjects. Advantageously, the reference profile may be a protein profile obtained from the patient herself, but at an earlier time, for example prior to treatment. Comparison of successive protein profiles for the patient, evaluating changes in biomarker expression, can yield valuable prognostic information and assist in subsequent treatment decisions. Alternatively, numerical values associated with one or more biomarkers in a test protein profile can be compared with reference values for the biomarkers, and deviations from the reference values may be indicative of disease state. Numerical values may represent raw, averaged or normalized values. The reference values may, but need not, be derived from the subject's own earlier protein profiles.

In another embodiment, the amount of at least one biomarker polypeptide in the test sample is compared with the amount of at least one other pre-identified polypeptide in the test sample, which serves as an internal standard. The pre-identified protein can be a biomarker for endometrial pathology, but is preferably not a biomarker for endometrial pathology. The relative difference or ratio (“test value”) between the biomarker polypeptide and the pre-identified “internal standard” polypeptide can be compared to a reference value that is indicative of endometrial disease status to determine whether the amount of at least one biomarker in the test sample indicates endometrial pathology.

In the present invention, selected peaks, and the polypeptides they represent, whether known or unknown, represent polypeptide biomarkers for endometrial pathology. Protein profiles or difference maps can be analyzed manually, if desired, but are preferably analyzed by computer. When little or no difference is observed between a reference pattern and a test sample pattern, the “difference” is indicative that the test sample is similar, as relates to the presence or absence of endometrial pathology, to the disease state represented by the reference profile. Alternatively, where there is a larger difference (e.g., 50% or more higher or lower than the reference) the test sample likely shares the disease state associated with the reference pattern.

Protein profiles can be analyzed and compared using commercially available or custom-made software. In a preferred embodiment, mass spectra are analyzed and compared using the ProteinChip Biomarker Wizard to identify potential biomarkers. Software for comparison of mass spectra are available in the art. For example, ProteinChip Software 3.1.1, designed for use with its ProteinChip Reader, is available from Ciphergen (Fremont Calif.). This software package performs comparisons of the mass spectra and identifies peaks that differ between samples.

Analysis software and protein array chips are also available from LumiCyte (Fremont, Calif.). Software designed for interpretation and comparison of mass spectrometry data is also available from, for example, ChemSW, Inc. (N. Fairfield, Calif.), Scientific Instrument Services (Ringoes, N.J.), Agilent Technologies (Palo Alto, Calif.), BioBridge Computing (Malmo, Sweden), and Bioinformatics Solutions (Waterloo, Ontario).

It should be understood that while mass spectrometric methods are preferred for generating protein profiles in accordance with the invention, protein profiles can be generated using any other suitable analytical technique such as two-dimensional gel electrophoresis, protein array analysis, population two-hybrid screening, and multiplexed immunoassay.

Illustrative Polypeptide Biomarkers for Endometrial Pathology

Illustrative polypeptide biomarkers associated with endometrial pathology, particularly endometrial cancer, are listed in Tables 3, 4, 5, 6 and 7 (Example I). These polypeptide biomarkers are represented as M/Z values (mass/charge ratios) which were identified in protein profiles generated using SELDI-TOF. These peaks are predictors of endometrial cancer; peak intensities that are higher or lower than those observed for a reference (normal/disease-free) sample are indicative of endometrial pathology. The peak values in these tables (in daltons) should be understood to include variation (tolerance) of at least ±one dalton, preferably at least ±five daltons, more preferably at least ±ten daltons. Alternatively, the variability of the M/Z values is at least about ±10%. The specific M/Z values, masses or molecular weights are not a critical parameter of the invention and may varying depending on the absorptive surface. Variations in experimental mass for the identified polypeptides are needed to reflect instrument-related accuracy and precision in obtaining M/Z values.

Tables 3, 4, 5, 6 and 7 (Example I) rank the specific proteins (in terms of M/Z values) found to be correlated (e.g., up- or down-regulated) with endometrial cancer. Preferred biomarkers are represented by M/Z values located within the top half of the list of biomarkers in Tables 3, 4, 5, 6 and 7; biomarkers that are more preferred are represented by the M/Z values located in the top quarter of the list, most preferably the biomarkers are represented by the top three or four M/Z values in the lists. The invention includes methods for identifying and further characterizing these individual biomarkers and others identified using methods described herein.

Classification Algorithms and Models

Protein profiles can also be further analyzed for patterns that allow classification of a sample based upon the pattern of expression of multiple biomarkers. Thus, in yet another embodiment, the invention provides a method for designing a classification algorithm or model that can be applied to a test protein profile to predict the disease state of the subject from whom the profile was obtained, wherein the disease state reflects the presence or absence of endometrial pathology. The invention further provides a method for predicting or assessing the disease state of a subject by applying a classification algorithm or model as described herein to the protein profile derived from a test sample, wherein the classification or model reflects patterns of biomarker expression that are associated with the presence or absence of endometrial pathology. Optionally the method further includes assigning scores of clinical sensitivity and specificity to the test sample.

A classification model is developed by identifying two classes of subjects, one with a known endometrial pathology, such as endometrial cancer, and one known (or assumed) to be free of the pathology. Biological samples are obtained from members of the two classes, protein profiles are produced either collectively or individually, and the protein profiles for the two classes are compared to identify polypeptides whose expression differ between the two classes. The protein profiles are preferably analyzed using software to identify hidden patterns of polypeptide expression that correlate with disease state.

The information content of the protein profiles can be elucidated and extracted using any of various computational algorithms, including algorithms commonly referred to as “artificial intelligence” algorithms. Classification models can be formed using any suitable statistical classification (or “learning”) method that attempts to segregate bodies of data into classes based on objective parameters present in the data, as described in detail in U.S. patent Pub. 20040096820 (published May 20, 2004, Rich et al.) and summarized here.

Classification methods may be either supervised or unsupervised. Examples of supervised and unsupervised classification processes are described in Jain et al., “Statistical Pattern Recognition: A Review”, IEEE Transactions on Pattern Analysis and Machine Intelligence, 2000, 2(1):4-37.

In supervised classification, “known” pre-classified samples are used to “train” a classification model. The data that are derived from the spectra and are used to form the classification model are referred to as a “training data set”. Once trained, the classification model can recognize patterns in data derived from spectra generated using unknown samples. The classification model can then be used to classify the unknown samples, for example to predict whether or not a particular biological sample is associated with an endometrial pathology. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines). Protein fingerprints specific for various cancers, including prostate, ovarian and breast cancers, have been derived from SELDI data using various computational algorithms. See, e.g., Adam et al., Cancer Res. 2002 62(13):3609-3614 (decision tree algorithm); Qu et al., Clin. Chem. 2002, 48(10)1835-1843 (decision tree algorithm); Petrocoin et al., Lancet 2002 359(9306):572-577 (genetic algorithm); and Li et al., Clin. Chem. 2002, 48(8):1296-1304 (support vector machine “SVM” algorithm).

A preferred supervised classification method is a recursive partitioning process. Recursive partitioning processes use recursive partitioning trees to classify spectra derived from unknown samples. The Biomarker Patterns Software (BPS) system (Ciphergen, Fremont Calif.) is an example of pattern recognition software for use in analyzing mass spectrometric protein profiles. This software can be used for further analysis of prospective peak SELDI-TOF biomarkers using a decision tree representation. A set of rules for organizing the samples according to phenotype is derived from analysis of the training and test spectral populations. Initially, a single splitting rule that best segregates the training set by phenotype is identified. The software then repeats the process on each resulting sub-classification of the data to produce a decision tree describing the best set of rules for organizing the samples according to phenotype. Preferably, the decision tree utilizes a splitting rule based on one or more of the biomarkers identified in Tables 3, 4, 5, 6 or 7 (Example I).

Decision tree analysis of SELDI mass spectral serum profiles for discriminating prostate cancer from benign conditions is reported in Qu et al. (Clin. Chem. 2002, 48:1835-1843). An analogous analysis for ovarian cancer is reported in Vlahou et al. (J. Biomed. Biotechnol. 2003, 5:308-314). Representative classification models for endometrial pathology using decision trees are described below in the Examples section. Further details about recursive partitioning processes are provided in U.S. patent Publ. 20020138208 Al (Paulse et al., Sep. 26, 2002) and WO 02/42733 (Paulse et al., May 30, 2002)). Additional learning algorithms for use in classifying biological information are described in, for example, WO 01/31580 (Barnhill et al., May 3, 2001); U.S. patent Publ. 20020193950 A1 (Gavin et al., Dec. 19, 2002); U.S. 20030004402 A1 (Hitt et al., Jan. 2, 2003); WO 02/06829 (Hitt et al., Jan. 24, 2002) and U.S. patent Publ. 20030055615 A1 (Zhang et al., Mar. 20, 2003).

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Optionally, patient history data and tumor biological characteristics are added to the classification algorithm or model to enhance the positive and negative predictive power of the classifier. The following clinical parameters are thus optionally included in the algorithm or model: patient age, race, phase of menstrual cycle, exposure to exogenous hormones, height, weight, and body mass index. Similarly, the following biological characteristics of tumors are optionally included in the algorithm or model to enhance the predictive value for recurrence of an existing endometrial cancer: tumor cell type (endometroid; clear cell, papillary serous), estrogen receptors alpha and beta, progesterone receptors A and B, androgen receptors, retinoic acid receptors, glucocorticoid receptors, epidermal growth factor receptors including HER-2/neu, HER-3, HER-4, insulin-like growth factor and its receptor(s), cytokines CSF-1, IL-1, IL-8, TNF-alpha, Ki-67, and apoptotic investigation as additional “nodes” in the algorithms: Ca-125, CEA (carcinoembryonic antigen), c-fms, and CSF-1.

Illustrative Classification Models

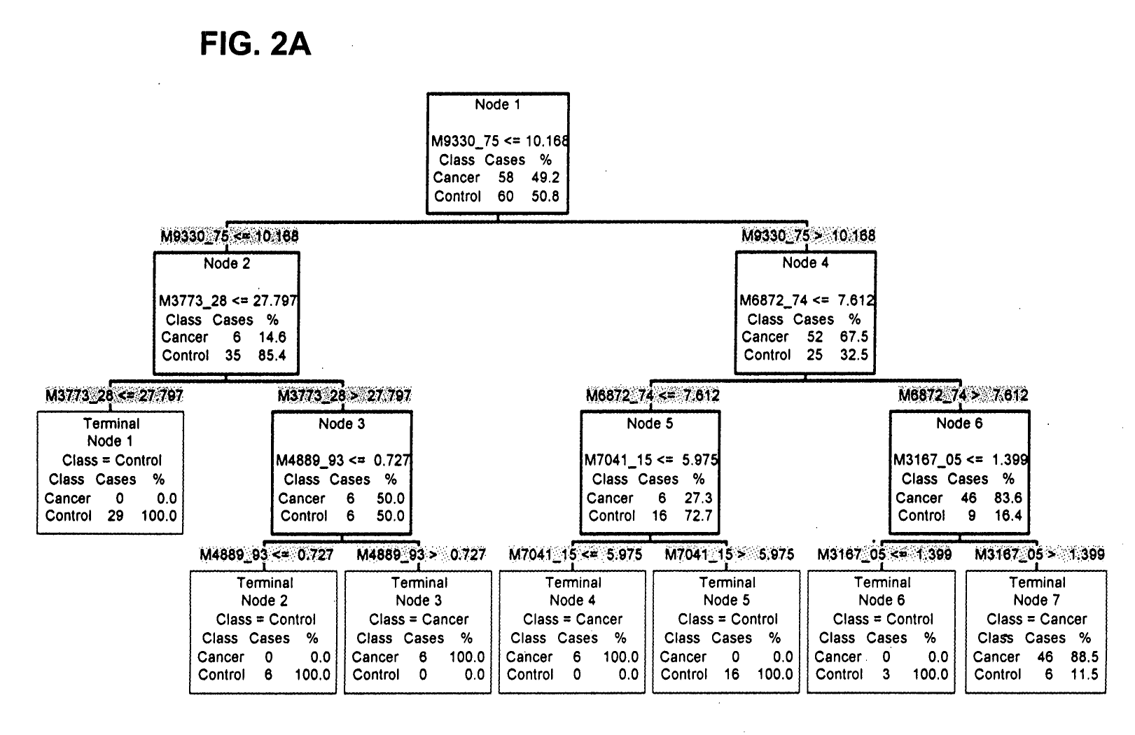

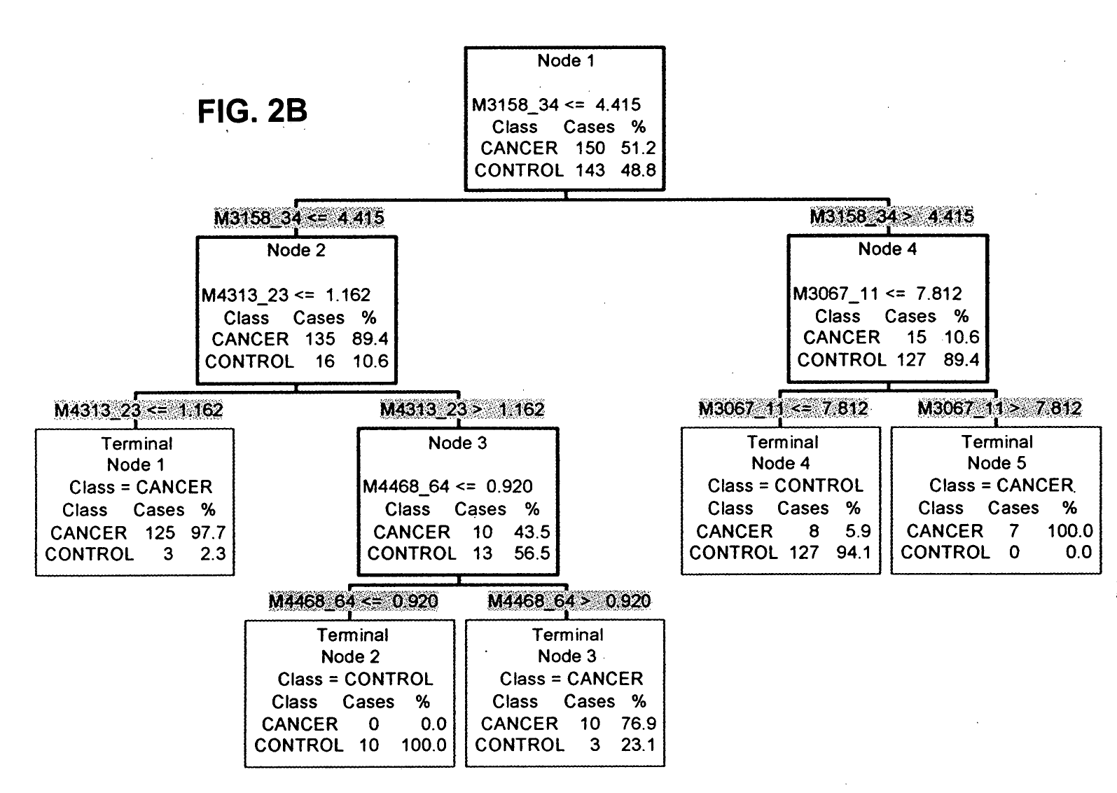

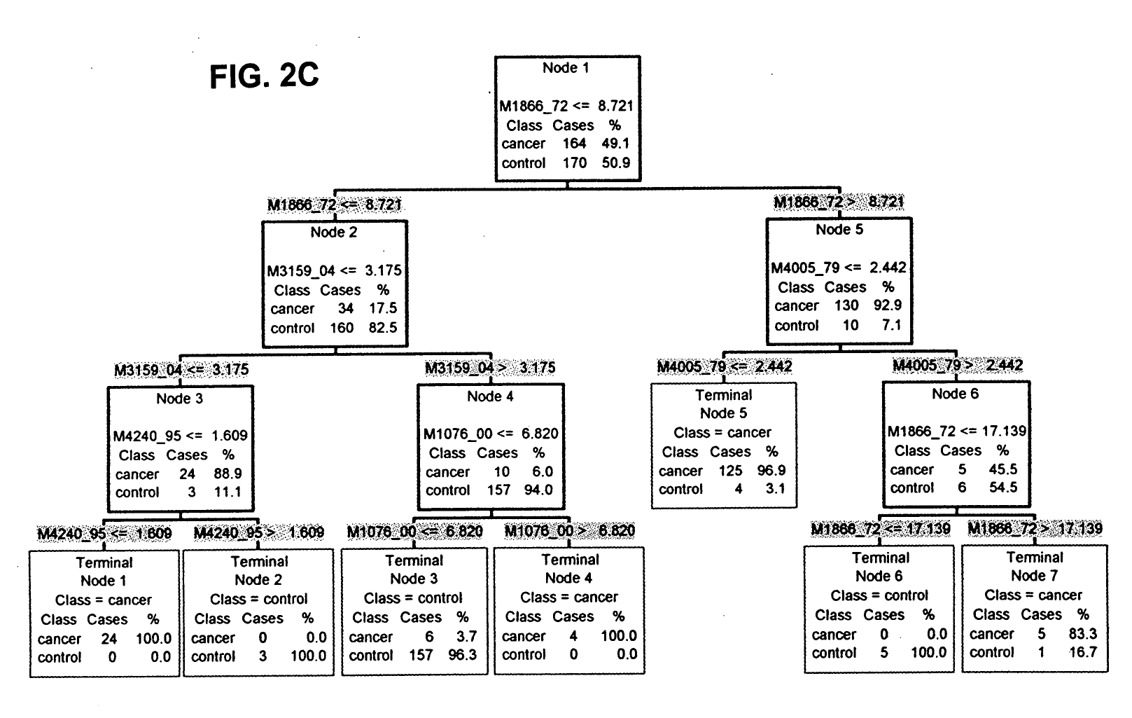

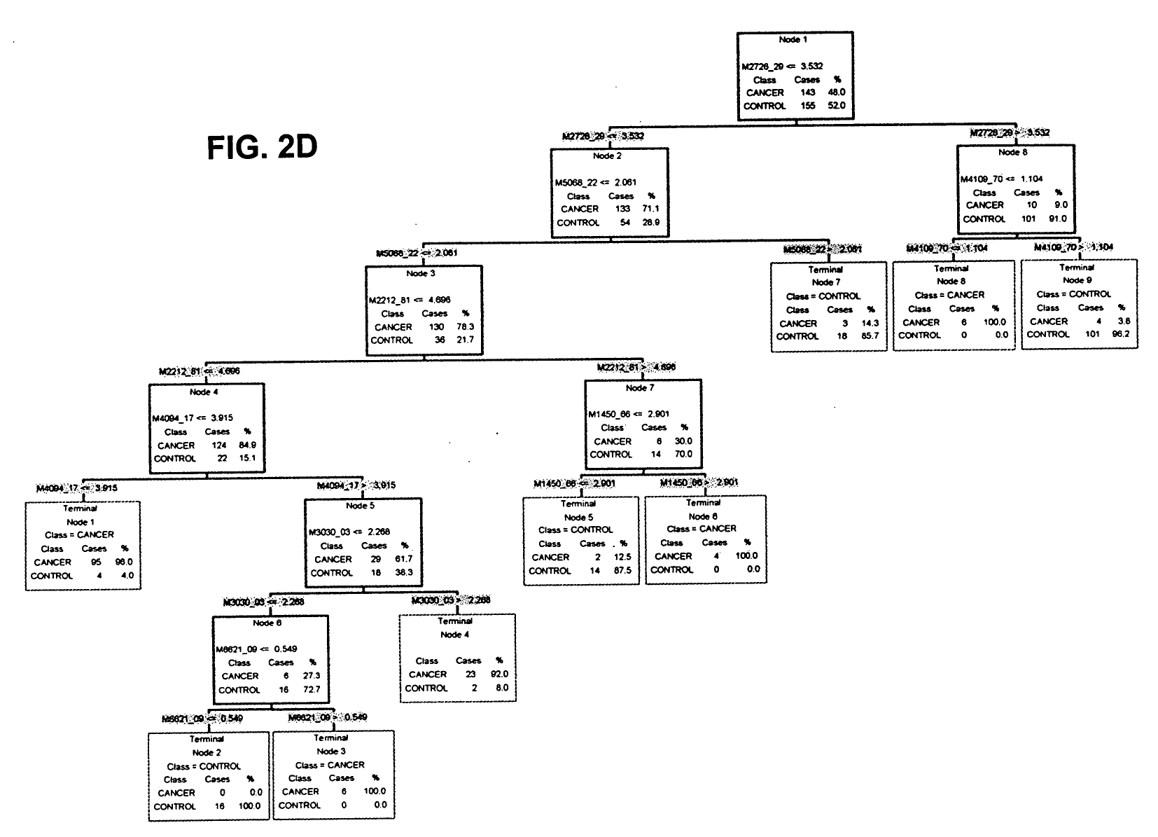

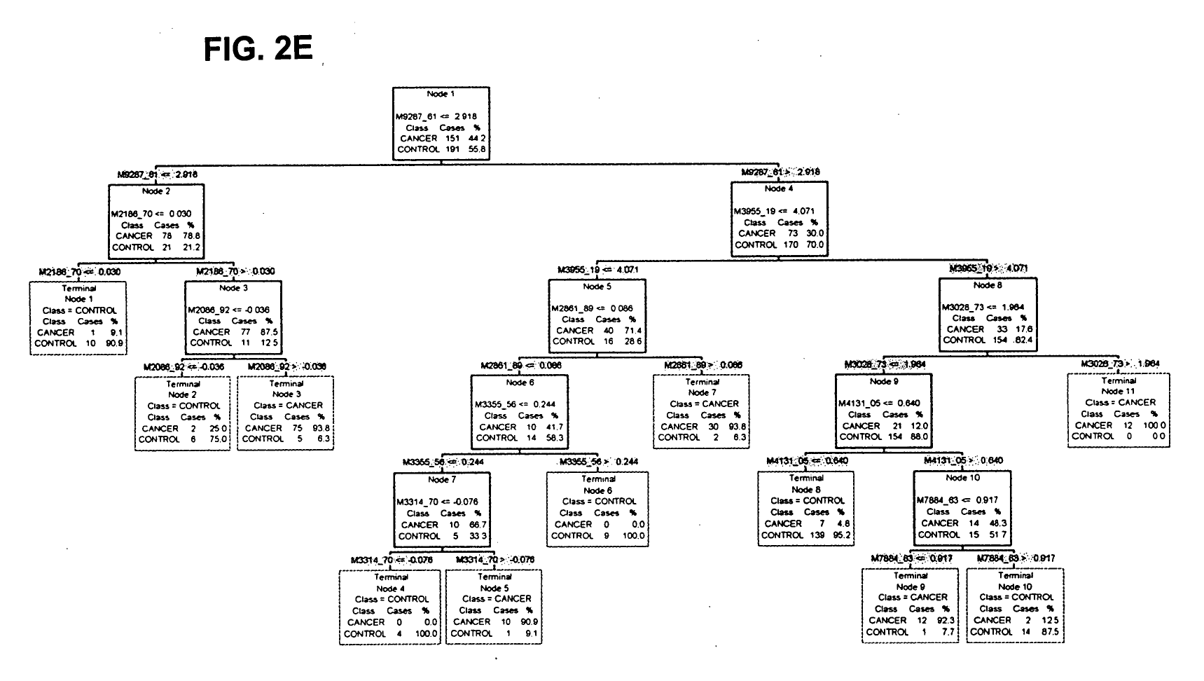

Illustrative classification models for use in assessing endometrial pathology are shown in FIG. 2 (Example I), and represent decision trees constructed using biomarkers selected from the lists in Tables 3, 4, 5, 6 and 7 (Example I). The M/Z values representing the biomarkers used in the decision tree are as follows:

| | |
|---|---|
| Table 3: | 9331, 3773, 4890, 6873, 7041, and 3167 |
| Table 4: | 3158, 4313, 4469, and 3067 |
| Table 5: | 1867, 3159, 4241, 1076, 4006, and 1867 |
| Table 6: | 2726, 5068, 2213, 4094, 3030, 6621, and 4110 |
| Table 7: | 9288, 2187, 3955, 2862, 3356, 3315, 3029, 4131, and 7885 |

Due to the variability in mass spectrometry data and the shifts in mass or molecular weight from a single peptide that is possible simply due to machinery settings, it is necessary to identify each peak as, for example, ±10%. Any one or more of these polypeptide peaks can be used in a classification model or algorithm according to the invention do discriminate pathological samples from nonpathological samples.
Development of Therapeutic Targets

The identification of biomarkers associated with disease can lead to the development of new therapeutic targets, as the protein underlying the biomarker peak can be identified and characterized. When an unknown biomarker is found to correlate closely with endometrial pathology, efforts can be focused on determining the identity of the biomarker polypeptide. Proteolytic peptide analysis and/or tandem mass spectrometry can be used to identify the protein, as can microsequencing technology.

The invention thus includes a method for identifying and characterizing a potentially therapeutic biomarker for endometrial pathology discovered using the methods described herein. A biomarker thus identified can be tracked to the particular sample fraction that contains it. The mass of the biomarker polypeptide is also known, as are one or more of the protein's binding affinities depending on the microchip chemistry that captured it. This allows the researcher to select and apply purification strategies appropriate for the particular biomarker. A purified protein can be sequenced using any convenient method, such as standard amino acid sequencing. Protein identification and/or sequencing can be accomplished using mass spectrometry, preferably tandem mass spectrometry (tandem MS). Although whole proteins can be analyzed using tandem MS, preferably the protein is fragmented prior to analysis. Using a system developed by Ciphergen (Fremont Calif.), peptide mass fingerprinting can be performed using the ProteinChip Biomarker System, followed by the transfer of the arrays to a ProteinChip Interface coupled to a tandem MS, for sequence verification. Method for identifying and characterizing polypeptide biomarkers, for example in connection with their development as therapeutic targets, are described in detail in U.S. patent Pub. 20040096820 (published May 20, 2004, Rich et al.).

Also included in the invention is a method for screening compounds for their stimulatory or inhibitory effect on a therapeutic target identified according to methods described herein.

Screening Assay

Also included is a method for screening a patient or a population of patients for endometrial pathology, particularly endometrial cancer. The method includes assaying for the presence of one or more biomarker polypeptides identified as described herein. An example is a simplified antibody-based screening test such as a Western blot or an enzyme linked immunosorbent assay (ELISA) which tests for the presence, absence or amount of a plurality of selected biomarkers associated with endometrial pathology. Preferably the assay tests for the presence, absence or amount of at least 2 polypeptides, preferably at least 3 or 4 polypeptides. Preferably, the assay tests for the presence, absence or amount of at most 12 polypeptides, more preferably at most 10 polypeptides, most preferably at most 8 polypeptides.

### EXAMPLES

The present invention is illustrated by the following examples. It is to be understood that the particular examples, materials, amounts, and procedures are to be interpreted broadly in accordance with the scope and spirit of the invention as set forth herein.

### Example I

### Identification of Peaks Associated With Endometrial Cancer

Summary

Mass spectrometry data were collected from serum samples from women with endometrial cancer and from controls. A candidate protein expression profile was identified that appears to distinguish early stage endometrial carcinoma from healthy controls. Peptide spectra were used to create algorithms with high sensitivity and high specificity in discriminating endometrial cancer.

Methods

Serum was collected in serum separator tubes. It is envisioned that in future work serum will be collected from all patients at diagnosis, at specific times during therapy, 6 months post-therapy, and at recurrence. At collection, the serum is spun in a low speed centrifuge at 1500 rpm for 3 minutes, and the serum is aliquoted into 1 ml cryovials and immediately frozen in liquid nitrogen. Samples will be forwarded to a central tissue collection facility for storage. At the laboratory, samples are thawed, a protease inhibitor added (Complete, Roche) separated into 10 μl aliquots, and refrozen. Serum samples are frozen at −70° C. until analysis. When ready to analyze, the serum samples are thawed on ice.

Mass spectrometry data were collected on a Ciphergen SELDI-TOF instrument using multiple ProteinChip arrays including the IMAC3 (metal affinity), the SAX2 (anion exchange), and the H50 (reversed phase hydrophobic). For the initial studies we found the H50 hydrophobic chip to be the most informative.

H50 protocol. The H50 (reversed phase hydrophobic) ProteinChip array was washed in 80% acetonitrile for 15-20 minutes. The chip was allowed to dry and 10 μl of binding buffer (50 mM KH₂PO₄ pH 7) was applied to the spot for 15 minutes. An aliquot (3 μl) of the sample was then added and mixed. This was left for an hour in a humidity chamber. The solution was then removed, using cotton swabs, and washed with 10 μl of binding buffer followed by a wash in water. The chip was then allowed to dry.

Protocol for the analysis of samples with anion and cation exchange protein arrays. The binding of proteins to anion and cation exchange chips is dependent on the pI of the protein and on the pH of the binding buffer. The cation exchange chips are shipped in sodium salt form and it is recommended to treat the chip with 10 mM HCl for 10 minutes before applying the binding buffer.

Optimization the pH for binding. The spots of the SAX2 (anion exchange) ProteinChip array were outlined using a mini pap hydrophobic pen to prevent diffusion of sample and contamination. This chip uses different pHs to optimize the pH for the samples. An aliquot (10 μl) of each pH buffer were added (pH's 9 to 3 buffer concentrations below) to the spots A-H respectively and incubated for 15 minutes. Then 3 μl of the sample was added, mixed and left for an hour in a humidity chamber. The solution was removed using cotton swabs and washed with the appropriate pH buffer, washed again with water and then the chip was allowed to dry.

Buffers used:

pH 9 buffer: 20 mM Tris .HCl
pH 8 buffer: 20 mM Tris .HCl
pH 7 buffer: 20 mM Na₂HPO₄/citric acid
pH 6 buffer: 20 mM Na₂HPO₄/citric acid
pH 5 buffer: 20 mM Na₂HPO₄/citric acid
pH 4 buffer: 20 mM Na₂HPO₄/citric acid
pH 3 buffer: 20 mM Na₂HPO₄/citric acid

Sax2 protocol. An aliquot (10 μl) of the selected pH 7 buffer was added to the spots and incubated for 15 minutes. The liquid was removed using cotton swabs and then another 10 μl of buffer was applied. 3 μl of sample was added, mixed and left for an hour in the humidity chamber. The solution was removed using cotton swabs and washed with the appropriate pH buffer and finally 10 μl of water. The chip was then allowed to dry.

Imac3 protocol (nickel protocol). The IMAC3 (metal affinity) ProteinChip array was soaked in 50 mM nickel (II) sulfate for 30 minutes. The chip was rinsed in distilled water to remove excess nickel sulfate. The chip was then soaked in a solution containing 0.1M sodium acetate/0.5M sodium chloride. The chip was removed and dried using cotton swabs. The rings of the IMAC3 ProteinChip array were outlined using a mini pap hydrophobic pen to prevent diffusion of sample. 10 μl of binding buffer was added to each spot. An aliquot (3 μl) of the sample was then added and mixed. This was left for an hour in a humidity chamber. After an hour, the solution was removed using cotton swabs and washed with binding buffer then water. The chip was then allowed to dry.

Preparation of the matrix solution. Cyano-4-hydroxycinnamic acid (CHCA) (5 mg, recrystallized) was weighed into an Eppendorf tube. Water (200 μl), acetonitrile (200 μl) and TFA (2 μl) was added and mixed. An aliquot (0.7 μl) of the CHCA matrix (100% saturated) was applied to each spot. The aliquots can then be stored at −20° C.

Protein chip analysis. All the chips were analyzed on the Protein Biology System 2 SELDI-TOF mass spectrometer (Ciphergen Biosystems, Fremont, Calif.). The ProteinChip arrays are 8 spot chips with 2 mm diameter spots. Serum from unaffected controls and patients with tumors were typically run concurrently on the same chip and on multiple chips. Peptides and proteins below the 30,000 mass/charge ratio were detected with α-cyano-4-hydroxy-cinnamic acid (CHCA) as a matrix, and analyzed with the Protein Biology System 2 SELDI-TOF mass spectrometer (Ciphergen Biosystems). For proteins above this range, sinapinic acid can be used as the matrix. SELDI is based on a MALDI-TOF format. Peaks of proteins correspond to a given mass and charge. Therefore, 2-4 peaks corresponding to multiple charges of an individual protein are often found.

Mass accuracy was assessed regularly using Ciphergen's All-in-one protein or peptide molecular weight standards. The internal controls provide a standard relative to all chips and allow peak height and/or presence to be taken into context. The All-in-1 peptide standard was used to ensure accurate peptide mass assignments. The peptides in this molecular weight standard include vasopressin (1.08 kDa), somatostatin (1.64 kDa), bovine B-chain (3.50 kDa), human insulin (5.81 kDa) and hirudin (7.03 kDa),

The ProteinChips were analyzed using the following instrument settings: laser intensity 170, detector sensitivity 8, focus lag time 950 ns, SELDI acquisition parameters 20, delta to 8, transients per to 10 ending position 80, molecular mass range optimized from 2000 to 20,000 Daltons. Instrument settings are further optimized for the mass range of proteins of interest. Data is collected and stored for later analyses.

Analysis of proteomics data combines elements from genetic algorithms and cluster analysis using Ciphergen proprietary software. The input data are ASCII files of proteomic spectra generated by SELDI-TOF. The Ciphergen Proteinchip software allows for relative comparison among peaks from treated, control, etc. It generates a statistics report that shows the average for each peak cluster and the p-value for each cluster. A cluster is a group of peaks that have similar masses, defined by a mass window (usually 0.3% mass error). Other peak measurements are resolution and peak area, intensities of peaks with similar masses and sample conditions as a group. The calculation used for each report will depend on the number of sample groups that have been selected. After selecting sample groups, visual comparisons of treated and control samples can be made to discern the differences among treated, tumor bearing, and control samples.

If differences are apparent, cluster information can be exported as a .csv file that can be read in the Biomarker Patterns Software (BPS) system (Ciphergen) for further analysis of prospective peak biomarkers. The Ciphergen Biomarker Patterns software was used to analyze all spectra from these experiments. This software package “learns” from a standard set of control samples (patterns) and allows for the identification of peaks and other subtleties of pattern recognition in samples. The Ciphergen Biomarker Patterns software finds hidden correlations to sample phenotypes identified by SELDI protein profiles. The software discovers patterns in the mass spectrometry data. Starting with SELDI peak intensity values from a “training set” of samples, Biomarker Patterns Software defines a single splitting rule that best segregates the training set by phenotype. The software repeats the process on each resulting sub-classification of the data to produce a decision tree describing the best set of rules for organizing the samples according to phenotype.

Data analysis was divided into two phases: 1) training and developing a model with known serum samples, and 2) testing the model with a separate set of known serum. Results were presented in an easy-to-interpret tree mode. The results also include assignment scores of clinical sensitivity and specificity. Once the software has been trained and the model generated, it can be utilized to classify “unknowns.” Patterns consisting of multiple biomarkers can be useful for clinical diagnosis.

Results

Initially, serum samples from 30 early stage endometrial cancer patients at a single institution were compared to 29 menopausal control volunteers with no previous history of cancer (Table 1). A decision tree model (not shown) was generated that discriminated between the cancer and control samples with 83% sensitivity and 80% specificity (Table 2a and 2b).

TABLE 1
Serum used for training set*

| Cancer Status | Number of Samples |
|---|---|
| No evidence of cancer | 29 |
| Biopsy proven endometrial cancer | 30 |
| Total | 59 |

*Control serum is from post-menopausal women without endometrial cancer.

TABLE 2a
Results for training set (H50 microarray).*

| Cancer Status | Samples | Misclassified | Pct Error | Cost |
|---|---|---|---|---|
| Control | 29 | 5 | 17.24 | 0.17 |
| Cancer | 30 | 12 | 20.00 | 0.20 |

*Model has 83% sensitivity and 80% specificity

TABLE 2b
Results for test set (H50 microarray).

| Cancer Status | Samples | Misclassified | Pct Error | Cost |
|---|---|---|---|---|
| Control | 6 | 0 | 0.00 | 0.00 |
| Cancer | 6 | 1 | 16.66 | 0.17 |

Refinement of Model and Validation

The addition of more samples to the training set increases the sensitivity and specificity of the initial discriminatory pattern. Additional samples were analyzed on the H50 microarray. The protein expression profile thus identified represents a clinically significant unique discriminatory pattern. The data (Table 3) are provided in the form of peaks, which represent mass and charge.

Table 3 lists biomarker polypeptides identified using the larger sample set. The peaks, indicated by a numeric descriptor preceded by an “M”, are described in terms of a ratio of mass to charge, i.e., M/Z.

TABLE 3
Biomarkers identified using the H50 microarray (in order of significance
with the most significant peak at the top of the list)

M9331
M7790
M10301
M8722
M6873
M8593
M7041
M3444
M8642
M8961
M5145
M5921
M3561
M4890
M3167
M3773
M6913
M22511
M1539
M2218
M2986
M17358
M3325
M3896
M3499
M6452

FIG. 2A shows the decision tree model generated from the larger sample set. In the graphical representations of the decision tree classification models in FIG. 2, the non-terminal nodes indicate the particular SELDI-TOF peak used to classify (split) the samples into two subgroups. As in the tables, the peak, indicated by a numeric descriptor preceded by an “M”, is described in terms in terms of a ratio of mass to charge, i.e., M/Z. The underscore represents a decimal point. The splitting rule, shown above each boxed node, is indicated in terms of a peak intensity for that peak. Peak intensities are shown as the log of the normalized intensities. Successive nonterminal nodes are shown until the splitting rules result in terminal nodes having high sensitivity and specificity for discriminating pathological (cancer) populations from normal (control) populations. The number of pathological (cancer) and normal (control) samples is shown in each node.

A series of characterization studies were conducted to determine which Ciphergen ProteinChip, column elution protocol and nitrogen laser intensity would be optimal using multiple ProteinChip arrays including the IMAC30 (copper metal affinity), the SAX2 (anion exchange), the WCX2, and the CM10. The IMAC30 chip provided the best resolution of multiple peaks over the 0 to 15,000 M/Z range with generally higher signal intensities.

A study using the IMAC30 ProteinChip was conducted to determine the extent to which each serum sample should be fractionated using Q Ceramic Hyper D F resin columns (BioSepra) and eluting proteins off with mediums of different pH. Based of the results of this study, we determined that we should elute proteins into three pH ranges: >pH 7, pH 7 to pH 5, and <pH 5. However, as is inherent with the SELDI format and most proteomics methods, no single protocol can be expected to capture a complete view of the proteome of interest, and one of skill in the art can readily carry out additional refinements.

Initially we fractionated the serum from 60 cancer patients and 60 control patients into three fractions. Subsequently additional samples were analyzed. Each elution fraction is stored frozen until the SELDI-TOF procedure. Spectra were obtained in triplicate for each fraction and an unfractionated serum sample. Using this platform, peptides and proteins below the 15,000 mass/charge ratio were detected with a-cyano-4-hydroxy-cinnamic acid as a matrix. Spectra were acquired and analyzed using the Ciphergen ProteinChip and Biomarker Patterns software. Statistically significant changes in multiple peak intensities between the mass spectra of the early stage endometrial cancer serum samples and controls were identified.

The protein expression profiles thus identified (Tables 4, 5, 6 and 7) represent clinically significant unique discriminatory patterns as described for Table 3 (the H50 data).

TABLE 4
Biomarkers identified using IMAC30 microarray,
pH < 5 fraction (in order of significance with
the most significant peak at the top of the list)
M3158
M4035
M3274
M4344
M4300
M3974
M4281
M3681
M4313
M3067
M4329
M3082
M2325
M4469
M8596
M3881
M2312
M2026
M2727
M8924
M3324
M3317
M8656

TABLE 5
Biomarkers identified using IMAC30 microarray,
pH 5-7 fraction (in order of significance with
the most significant peak at the top of the list)
M1867
M1027
M4018
M1930
M2025
M9005
M1887
M3159
M3973
M3990
M4282
M4035
M4006
M3292
M1076
M2789
M2311
M2726
M5012
M3068
M4241
M4006
M2053
M5395
M4648
M1156
M1531
M3957
M3275

TABLE 6
Biomarkers identified using IMAC30 microarray,
pH > 7 fraction (in order of significance with
the most significant peak at the top of the list)
M2726
M2030
M2093
M3337
M3355
M3273
M5068
M3030
M2882
M9280
M4110
M2273
M2213
M4094
M3510
M2250
M6621
M4078
M3810
M7561
M5857
M8907
M9030
M8945
M1451
M5132
M3971
M9342
M2368
M1841
M1780
M4644
M1946
M4034
M3955
M4666
M4054

TABLE 7
Biomarkers identified using IMAC30 microarray, nonfractionated
(in order of significance with the most significant
peak at the top of the list)
M9288
M3955
M7768
M1533
M3029
M3974
M1595
M4300
M4503
M4656
M2953
M7885
M7816
M2187
M4131
M4281
M4017
M7751
M3995
M9341
M4018
M3275
M5341
M5912
M2087
M2273
M2862
M5970
M2726
M4433
M3356
M2026
M3315
M5330
M8958
M4038
M4643
M2012
M9419
M5931
M2397
M2985
M2211
M1657

Table 8 summarizes the sensitivity and specificity of models developed from each fraction. A protein expression profile was identified from the pH 5 fraction that distinguishes early stage endometrial carcinoma from healthy controls with 94% sensitivity and 93% specificity. Decision tree classification models for the various pH fractions are shown in FIG. 2B-2E.

TABLE 8
Training Sets models developed from different pH fractions

| Fraction | Sensitivity | Specificity |
|---|---|---|
| NonFractionated | 81% | 82% |
| ≧pH 7 | 89% | 87% |
| pH 7 to pH 5 | 94% | 93% |
| ≦pH 5 | 94% | 89% |

The results from each model can be combined to further refine the model and increase the sensitivity and specificity. After determining the refined pattern, validation can be performed using a blinded set of healthy and cancer patients. As each patient sample is examined and categorized, it can be added to the model, strengthening and further refining it. Subsequently, the origin and full identity of the discriminating proteins can be determined, as described in Example II.

These endometrial cancer predictor peaks should be understood to include the different potential experimental mass variations for each individual protein. The peaks at the identified M/Z values (±10%), and the polypeptides identified at these molecular weights, function as discriminators for the diagnosis of endometrial cancer, pre-cancer (endometrial hyperplasia of any type), endometriosis, and/or other diseases of the endometrium in patient sera. Due to the variability in mass spectrometry data and the shifts in mass or molecular weight from a single peptide that is possible simply due to machinery settings, it is justifiably advisable to identify each peak ±10%.

### Example II

### Identification of Particular Polypeptide Biomarkers

In Example I, whole serum samples were fractionated using Q Ceramic Hyper DF sorbent columns. Any potential discriminatory biomarkers discovered will have already been assigned to a known pH fraction on the IMAC 30 ProteinChip Array surface. Using this knowledge, a marker can be first purified using Q HyperD F column chromatography, then the pH fraction of interest can be purified to enrich for the biomarker. The fraction containing the marker is purified through a second chromatography step using IMAC HyperCel Spin Columns (Ciphergen, Fremont Calif.), which have a matched chemistry to IMAC 30. The marker is additionally purified, concentrated and desalted using a reversed phase step. Finally the protein of interest is purified using one dimensional SDS PAGE.

Following in-gel digestion, protein identification can be attempted by MALDI Time of Flight Mass Spectrometry (MALDI-TOF) using an AP Biosystems Voyager Elite instrument. Proteins not identified by MALDI-TOF can be identified with electrospray (ESI) MS-MS with a high resolution Q-Tof mass analyzer (Micromass Q-Tof2 ESI mass spectrometer, Waters Corp.). These two ionization techniques are complementary because they are known to produce different MS and MS/MS spectra from the analysis of the same sample due to the differential ionization of peptides. Samples are introduced into the Q-Tof via a Micromass CapLC (Waters Corp.) that is an automated solvent/sample management system specifically for integration with Q-Tof. The Micromass ProLynx software (Waters Corp.) automatically performs database searches with peptide and sequence data to identify proteins.

Alternative methods of protein isolation and identification using 2-D gels can be used if desired or necessary. For these experiments, mass spectrometry linked to 2-dimensional gel electrophoresis is performed on serum peptide extracts. Peaks of interest are isolated from large volumes of fractionated serum. Sera is obtained from pooled samples of appropriate specimens. Proteins are extracted using the ReadyPrep sequential extraction kit (BioRad) where differential solubilization can be utilized to reduce sample complexity. For running 2-D gels, n IPGphor (Amersham Pharmacia Biotech) is utilized for first dimension separation of proteins on pre-cast immobilized pH gradient gels (IPG strips). Second dimensional SDS-PAGE electrophoresis is performed on two different size formats. For preliminary analysis, the Bio-Rad Criterion electrophoresis Dodeca Cell uses 11 cm IPG strips and runs up to 12 gels simultaneously. Preparatory large format gels can be used to run samples if increased sample concentration is needed for identification. The protein separation facility uses a Hoefer DALT with 18 cm IPG gel strips and can run up to 12 large format (23×20 cm) gels. Imaging is accomplished with a Bio-Rad GS-800 calibrated densitometer. Imaged gels are analyzed and databased by PD Quest software (Biorad). Patterns of differential protein expression are identified by the software and proteins of interest excised and subjected to in-gel enzymatic digest to extract the peptides.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method for evaluating the presence, absence, nature or extent of an endometrial pathology in a patient comprising:
detecting a plurality of polypeptides in a biological test sample obtained from the patient to yield a test protein profile; and
comparing the test protein profile with a reference protein profile; wherein a difference between the test protein profile and the reference protein profile is indicative of the presence, absence, nature or extent of the endometrial pathology in the patient.

2. The method of claim 1 wherein the reference protein profile represents at least one biomarker polypeptide.

3. The method of claim 1 wherein the reference protein profile represents a plurality of biomarker polypeptides.

4. The method of claim 1 wherein the difference between the test protein profile and the reference protein profile comprises a difference in the amount of at least one biomarker polypeptide represented by a M/Z peak value in Tables 3, 4, 5, 6 or 7.

5. The method of claim 1 wherein the comparing step comprises discriminating between different disease states or between a disease state and normal state.

6. The method of claim 1 wherein the difference between the test protein profile and the reference protein profile is indicative of the progression or regression of endometrial pathology in the patient.

7. The method of claim 6 wherein the reference protein profile is derived from a sample previously obtained from the patient.

8. The method of claim 1 wherein the comparing step comprises evaluating or monitoring the efficacy of treatment of the patient.

9. The method of claim 1 further comprising designing a classification model or algorithm based on at least one difference between the test protein profile and the reference protein profile.

10. A method for evaluating the presence, absence, nature or extent of an endometrial pathology in a patient comprising:
detecting a plurality of polypeptides in a biological test sample obtained from the patient to yield a test protein profile showing the amount of at least one biomarker polypeptide in the sample; and
comparing the amount of the biomarker polypeptide in the sample with at least one predetermined reference value;
wherein a difference between the amount of the biomarker polypeptide in the sample and the predetermined reference value is indicative of the presence, absence, nature or extent of the endometrial pathology in the patient.

11. The method of claim 10 wherein the test protein profile is generated using mass spectrometry, and the amount of the biomarker polypeptide is indicated as a spectral peak intensity.

12. The method of claim 10 where the biomarker polypeptide is represented by a M/Z peak value in Tables 3, 4, 5, 6 or 7.

13. A method for evaluating the presence, absence, nature or extent of an endometrial pathology in a patient comprising:
detecting at least one biomarker polypeptide in a biological test sample obtained from the patient;
detecting at least one reference polypeptide in the test sample;
comparing the amount of the biomarker polypeptide to the amount of the reference polypeptide in the test sample to yield a test value; and
comparing the test value to a predetermined reference value;
wherein a difference between test value and the predetermined reference value is indicative of the presence, absence, nature or extent of the endometrial pathology in the patient.

14. The method of claim 13 wherein the biomarker polypeptide is represented by a M/Z peak value in Tables 3, 4, 5, 6 or 7.

15. A method for evaluating the presence, absence, nature or extent of an endometrial pathology in a patient comprising:
detecting a plurality of polypeptides in a biological test sample obtained from the patient to yield a test protein profile; and
analyzing the test protein profile using a classification model or algorithm to discriminate the presence, absence, nature or extent of the endometrial pathology in the patient;
wherein the model or algorithm is derived from analysis of a plurality of protein profiles known to be associated with the presence, absence, nature or extent of the endometrial pathology.

16. The method of claim 15 wherein the analysis of the plurality of protein profiles is made using supervised or unsupervised learning methods.

17. The method of claim 15 wherein the analysis of the plurality of protein profiles is made using a recursive partitioning process.

18. The method of claim 15 wherein the model is a decision tree model.

19. The method of claim 15 wherein the model or algorithm discriminates on the basis of the presence, absence or amount of at least one biomarker polypeptide having m/z listed in Tables 3, 4, 5, 6 and 7.

20. The method of claims 1, 10, 13 or 15 further comprising immobilizing the plurality of polypeptides on a microarray prior to detecting the polypeptides.

21. The method of claim 20 wherein the plurality of polypeptides is detected using surface-enhanced laser desorption/ionization time of flight (SELDI-TOF) mass spectrometry.

22. The method of claims 1, 10, 13 or 15 wherein the endometrial pathology comprises endometrial cancer, hyperplasia or endometriosis.

23. The method of claims 1, 10, 13 or 15 wherein the biological sample comprises blood, serum or vaginal secretions.

24. A computer-assisted method for evaluating the presence, absence, nature or extent of an endometrial pathology in a patient comprising:
providing a computer comprising model or algorithm for classifying data from a biological sample obtained from a subject, wherein the classification includes analyzing the data for the presence, absence or amount of at least one biomarker polypeptide;
inputting data from a biological sample obtained from a subject; and
classifying the biological sample to indicate the presence, absence, nature or extent of an endometrial pathology.

25. The method of claim 24 wherein the biomarker polypeptide is represented by a M/Z peak value in Tables 3, 4, 5, 6 or 7.

26. A method for identifying a polypeptide biomarker associated with the presence, absence, nature or extent of an endometrial pathology comprising:
detecting a plurality of polypeptides in a biological test sample obtained from the patient to yield a test protein profile;
comparing the test protein profile with a reference protein profile, wherein a difference between the test protein profile and the reference protein profile is indicative of the existence of a biomarker polypeptide associated with the presence, absence, nature or extent of endometrial pathology in the patient; and
identifying the polypeptide biomarker.

27. A method for identifying a polypeptide biomarker associated with the presence, absence, nature or extent of an endometrial pathology comprising:
detecting a plurality of polypeptides in a first plurality of biological samples obtained from test patients known to be afflicted from an endometrial pathology to yield a test protein profile;
detecting a plurality of polypeptides in a second plurality of biological samples obtained from control patients known to be free of the endometrial pathology to yield a control protein profile; and
comparing the test and control protein profiles to identify a polypeptide biomarker associated with the presence, absence, nature or extent of the endometrial pathology.

28. The method of claim 26 or 27 further comprising isolating the biomarker polypeptide.

29. The method of claim 26 or 27 further comprising determining the amino acid sequence of the biomarker polypeptide.

30. The method of claim 26 or 27 further comprising evaluating the suitability of the biomarker polypeptide as a therapeutic target.

31. The method of claim 30 wherein the biomarker polypeptide comprises a therapeutic target, the method further comprising screening compounds for efficacy in altering the bioactivity of the biomarker polypeptide.

32. A method for detecting biomarker polypeptides associated with endometrial pathology, the method comprising:
producing a protein profile from a test sample obtained from a subject suspected of having an endometrial pathology;
comparing the protein profile of the test sample with a reference protein profile that is indicative of the presence or absence of the endometrial pathology; and
detecting differentially expressed polypeptides between the first and second profiles, wherein said differentially expressed proteins are biomarker polypeptides.

33. The method of claim 32 further comprising evaluating the presence or absence of an endometrial pathology in a patient in view of the expression of at least one biomarker polypeptide.

34. The method of claim 32 further comprising isolating and identifying at least one biomarker polypeptide.

35. A method for screening a patient or population of patients for endometrial pathology comprising:
assaying a biological sample obtained from a patient for the presence of at least one biomarker polypeptide associated with endometrial pathology.

36. The method of claim 35 wherein the assay comprises a mass spectrometric assay.

37. The method of claim 35 wherein the assay comprises an immunoassay.

38. The method of claim 37 wherein the immunoassay comprises a Western blot or an enzyme linked immunosorbent assay.

39. The method of claim 35 comprising analyzing the biological sample for a plurality of biomarker polypeptides.

40. The method of claim 35 wherein the biomarker peptide is represented by a mass spectrometric peak value in Tables 3, 4, 5, 6 or 7.
